(19) United States
(12) Patent Application Publication
(10) Pub. No.: US 2021/0213150 A1
(43) Pub. Date: Jul. 15, 2021

(54) INTEGRATED STERILIZATION CONSUMABLES FOR LOAD MONITORING

(71) Applicant: Accelera Technologies, LLC, Minneapolis, MN (US)

(72) Inventor: Jacob R. Gibbons, Minneapolis, MN (US)

(21) Appl. No.: 17/130,093

(22) Filed: Dec. 22, 2020

Related U.S. Application Data
(60) Provisional application No. 62/958,775, filed on Jan. 9, 2020.

(51) Int. Cl. A61L 2/28

(52) U.S. Cl. CPC A61L 2/28; A61L 2202/24; A61L 2202/14; A61L 2202/122

(57) ABSTRACT

A cassette for use in a decontamination system that delivers sterilant for decontaminating one or more loads and includes at least one process challenge device (PCD), such as a biological and/or chemical indicator. The decontamination system includes a cassette receiving area to receive the cassette such that at least a portion of the cassette is in fluid communication with the interior of the decontamination chamber. During the decontamination process, sterilant fluid from the cassette is delivered to decontaminate a load. The PCD in the cassette is used to confirm effective sterilization of the device to be decontaminated.

### CROSS REFERENCE TO RELATED APPLICATIONS

This application claims priority and benefit of U.S. Provisional application with Ser. No. 62/958,775, filed Jan. 9, 2020, entitled INTEGRATED STERILIZATION CONSUMABLES FOR LOAD MONITORING, which is herein incorporated by reference in its entirety.

### TECHNICAL FIELD

This disclosure relates generally to decontamination of medical devices; in particular, this disclosure relates to a cassette for a decontamination system that delivers sterilant and includes a process challenge device (PCD) to confirm effective sterilization of the device that was decontaminated.

### BACKGROUND

Robust medical instruments are often sterilized at high temperatures. Commonly, the instruments are sterilized in a steam autoclave under a combination of high temperature and pressure. While such sterilization methods are very effective for more durable medical instruments, advanced medical instruments formed of rubber and plastic components with adhesives are delicate and wholly unsuited to the high temperatures and pressures associated with a conventional steam autoclave.

Steam autoclaves have also been modified to operate under low pressure cycling programs to increase the rate of steam penetration into the medical devices or associated packages of medical devices undergoing sterilization. Steam sterilization using gravity, high pressure or pre-vacuum create an environment where rapid changes in temperature can take place. In particular, highly complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

In some circumstances, a process challenge device (PCD), such as a chemical or biological indicator, will be added to a decontamination load to determine decontamination effectiveness. This requires separate user interaction with the decontamination system, and can result in user errors. For example, the user could forget to add the PCD when loading the device to be decontaminated or choose the wrong PCD for the type of device. Additionally, in some cases, PCD devices can be assembled and prepared by healthcare facilities, which can lead to inconsistencies and added risk in efficacy and resistance of the PCD device.

Therefore, a need exists that overcomes one or more of the disadvantages of present decontamination systems.

### SUMMARY OF THE INVENTION

According to one aspect, this disclosure provides a decontamination system with a decontamination chamber, a cassette, and a sterilization control system. The decontamination chamber has a plurality of walls defining a device receiving area dimensioned to receive a device to be decontaminated; at least one wall of the plurality of walls define a cassette receiving area in fluid communication with the device receiving area. The cassette is dimensioned to be detachably received by the cassette receiving area, wherein the cassette includes (i) a sterilant delivery portion to deliver a sterilant to the device receiving area; and (ii) a process challenge device (PCD) portion to confirm effective sterilization of the device to be decontaminated, wherein at least a portion of the sterilant delivery portion and at least a portion of the PCD portion is in fluid communication with the device receiving area when the cassette is attached to the cassette receiving area. The sterilization control system is to control sterilant delivery from the sterilant delivery portion to the device receiving area.

According to another aspect, this disclosure provides a cassette for use with a decontamination system. The cassette includes a cassette body defining at least one internal sterilant reservoir and at least one internal PCD reservoir; a volume of sterilant within the at least one internal sterilant reservoir; at least one indicator relevant to sterilization of a device to be decontaminated within the at least one internal PCD reservoir. The cassette body defines an outlet port to deliver at least a portion of the sterilant from the internal sterilant reservoir and an inlet port to provide fluid communication with the internal PCD reservoir.

### BRIEF DESCRIPTION OF THE DRAWINGS

The present disclosure will be described hereafter with reference to the attached drawings which are given as non-limiting examples only, in which:

FIG. 1 is diagrammatic view of a system for decontaminating a lumen device according to an embodiment of the present disclosure;

FIG. 2 is a diagrammatic view of an example cassette that delivers sterilant for decontaminating a lumen device and includes a process challenge device (PCD) according to an embodiment of the present disclosure;

FIG. 3 is a diagrammatic view of an example cassette that delivers sterilant for decontaminating multiple lumen devices and includes a process challenge device (PCD) according to another embodiment of the present disclosure; and

FIG. 4 is a diagrammatic view of an example cassette that delivers sterilant for decontaminating multiple lumen devices and includes multiple process challenge devices (PCDs) according to another embodiment of the present disclosure.

Corresponding reference characters indicate corresponding parts throughout the several views. The components in the figures are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure.

This disclosure relates to a cassette that can be placed in fluid communication with the decontamination chamber of a decontamination system and delivers sterilant for decontaminating one or more loads (which could be a mixed load, a non-lumen load, and/or a lumen load, etc.) and includes at least one process challenge device (PCD). In some embodiments, the decontamination system includes one or more walls that are configured to receive the cassette such that at least a portion of the cassette is in fluid communication with the interior of the decontamination chamber. During the decontamination process, sterilant fluid from the cassette is delivered to decontaminate the load. The PCD in the cassette is used to confirm effective sterilization of the load to be decontaminated. The cassette defines a tortuous path between the indicator and the device receiving area. In some embodiments, the cassette could include sterilant fluid and a PCD for a single decontamination load or cycle; in other embodiments, the cassette could include sterilant for multiple loads/cycles and/or multiple PCDs. Depending on the circumstances, the decontamination system could include a built-in monitoring system that is configured to read the cassette's biological indicator (BI) and/or chemical indicator (CI) to determine whether the lumen device was effectively decontaminated.

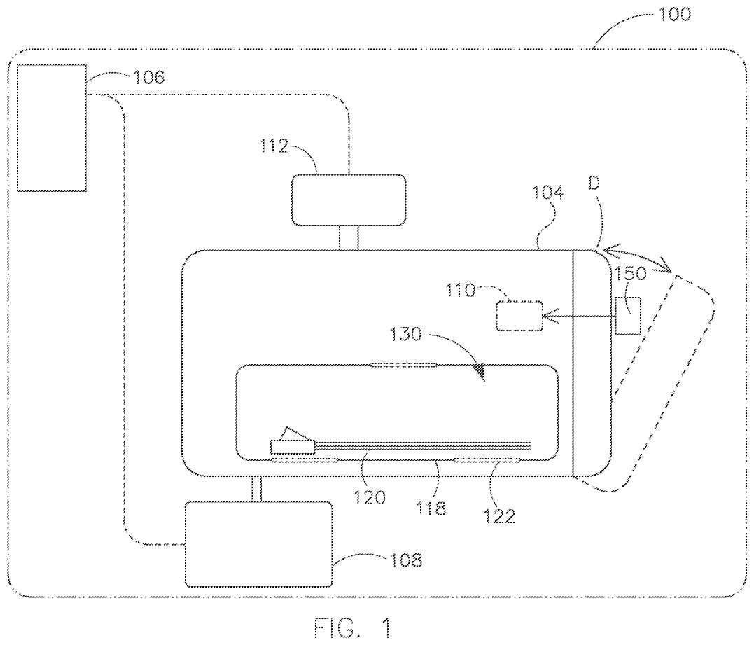

FIG. 1 is a diagrammatic view of one embodiment of a system 100 for decontaminating a load, which is broadly intended to mean any device to be decontaminated, including but not limited to a medical, dental, or other device having one or more lumens extending there-through and/or a medical, dental, or other device without any lumens. The system 100 includes a decontamination chamber 104, a system controller 106, an environmental monitoring and control system 108, a cassette receiving area 110 and a PCD monitoring system 112 configured to read at least one chemical and/or biological indicator of a PCD to determine effectiveness of sterilization. The cassette receiving area 110 is dimensioned to receive a cassette 150 such that at least a portion of the cassette 150 is in fluid communication with the interior of the decontamination chamber 104. The cassette 150 includes sterilant fluid and one or more PCDs. During decontamination, the decontamination system 100 is configured to deliver sterilant fluid within the cassette 150 to the decontamination chamber 104. The one or more PCDs within the cassette 150 include chemical and/or biological indicators that can be used to determine sterilization effectiveness. The PCD monitoring system 112 is configured to read the PCD in the cassette 150 to determine sterilization effectiveness, which could be displayed or otherwise indicated by the decontamination system 100. For example, the PCD monitoring system 112 could potentially visually analyze sterilant flowing from the cassette 150 to determine an injection rate and/or other information. In some embodiments, the PCD monitoring system 112 could analyze the PCD and provide status information regarding the PCD. When a chemical indicator is used, for example, the PCD monitoring system 112 could indicate unchanged/unprocessed/fail or changed/processed/pass. By way of another example, when a biological indicator is used, the PCD monitoring system could indicate the growth indicator, florescence or information (e.g., growth/fail or no growth/pass). In some embodiments, the PCD monitoring system 112 could be optional and an external PCD reader separate from the decontamination system 100 could be used to read the cassette's PCD.

In some embodiments, a terminal package 118 containing a load 120 for decontamination may be placed within the decontamination chamber 104. Depending on the circumstances, the terminal package 118 could be optional, and the load could be decontaminated by placing it in the receiving area 110. In the illustrated embodiment, the terminal package 118 includes a fluid inlet, which could be in the form of a plurality of openings or pores 122. The cassette receiving area 110 may be in fluid communication with the decontamination chamber 104, which allows sterilant fluid within a cassette 150 placed in the cassette receiving area 110 to flow within the interior of the decontamination chamber 104 for decontaminating the load 120. Although FIG. 1 shows the cassette receiving area 110 within a side wall of the decontamination chamber 104, the cassette receiving area 110 could be located on the top, bottom or any wall of the decontamination chamber 104.

The system controller 106 provides control signals to and/or receives condition sensing and equipment status signals from the decontamination chamber 104, environmental monitoring and control system 108, and/or the PCD monitoring system 112. In some embodiments, the system 100 can be assembled in a device small enough to sit on a tabletop or counter. For example, the decontamination chamber 104 may have an interior volume of less than about ten cubic feet.

The load 120 to be decontaminated can be placed into the decontamination chamber 104 by opening the door D and placing the load 120 on a rack or other supporting assembly in the interior of the decontamination chamber 104. In some embodiments, the load 120 may be enclosed in the terminal package 118 before being placed in the decontamination chamber 104. In the example shown, the terminal package 118 defines a load receiving area 130 to receive the load 120 for decontamination. In the illustrated embodiment, the terminal package 118 includes a plurality of openings or pores 122.

As discussed above, the cassette 150 may include one or more cavities configured to hold a sterilant fluid. In some embodiments, the sterilant fluid can be a chemical or other substance suitable for use in a sterilization process that complies with the American National Standard (ANSI) standard ANSI/AAMI ST56:2013, “Chemical Sterilization and High-level Disinfection in Health Care Facilities.” In some embodiments, the sterilant fluid 156 can be a room temperature (e.g., 20° C. to 25° C.) substance that can be dispersed as a fluid, such as a liquid, a vapor, or a combination thereof (such as a fog) during the decontamination process. Suitable substances for the sterilant fluid include hydrogen peroxide (H₂O₂) and peracetic acid (PAA).

In various embodiments, the sterilant fluid is a composition that includes: (a) hydrogen peroxide; (b) organic acid; (c) a polymeric sulfonic acid resin based chelator; and (d) surfactant. The composition includes less than about 1 wt. % of an anticorrosive agent. The composition can further optionally include water.

In one aspect, the hydrogen peroxide present in the composition can be from about 0.5 wt. % to about 30 wt. %, from about 0.5 wt. % to about 1.5 wt. %, from about 0.8 wt. % to about 1.2 wt. %, from about 20 wt. % to about 30 wt. % and all ranges and values from about 0.5 wt. % to about 30 wt. %.

In another aspect, the acetic acid present in the composition can be from about 1 wt. % to about 25 wt. %, from about 4 wt. % to about 20 wt. %, from about 4.5 wt. % to about 5.5 wt. %, from about 9 wt. % to about 17 wt. % and all ranges and values from about 1 wt. % to about 25 wt. %.

In still another aspect, the peracetic acid present in the composition can be from about 0.01 wt. % to about 25 wt. %, from about 0.05 wt. % to about 20 wt. %, from about 0.05 wt. % to about 0.1 wt. %, from about 3.5 wt. % to about 8 wt. % and all ranges and values from about 0.01 wt. % to about 25 wt. %.

In yet another aspect, the polymeric resin chelator present in the composition can be from about 0.1 wt. % to about 5 wt. %, from about 0.2 wt. % to about 2 wt. %, from about 0.5 wt. % to about 1.5 wt. % and all ranges and value from about 0.1 wt. % to about 5 wt. %.

In various embodiments, the present invention provides for a composition that includes: (a) hydrogen peroxide, present in a concentration of about 0.5 wt. %to about 30 wt. %, e.g., about 28 wt. %; (b) acetic acid, present in a concentration of about 3 wt. % to about 25 wt. %, e.g., about 16 wt. %; (c) a sulfonic acid supported polymeric resin chelator present in a concentration of about 0.1 wt. % to about 5 wt. %, e.g., about 0.2 wt. % to about 0.7 wt. %; and, optionally, (d) Pluronic® 10R5 surfactant block copolymer, present in a concentration of about 2.0 wt. %, wherein the composition comprises less than about 0.1 wt. % of an anticorrosive agent, e.g., 0 wt. % of an anticorrosive agent. The composition can further optionally include water. In some embodiments, the hydrogen peroxide and acetic acid can combine to form peracetic acid, present in about 4 wt. % to about 8 wt. %, e.g., 6.8-7.5 wt. %.

In certain aspects, the peracetic acid/hydrogen peroxide compositions are stabilized without the need for a phosphonic based chelator, such as 1-hydroxyethylidene-1,1,-diphosphonic acid. In other aspects, a phosphonic based chelator, such as 1-hydroxyethylidene-1,1,-diphosphonic acid can be included in the sterilant fluid and therefore, component c), the polymeric sulfonic acid resin is optional. This is detailed in pending U.S. application 62/737,453, filed Sep. 27, 2018, entitled “Peracetic Acid Stabilized Compositions with Polymeric Resins Chelators”, the contents of which are incorporated herein by reference.

The terminal package 118 is sized so that the load 120 to be decontaminated fits within the terminal package 118. In some embodiments, the terminal package 118 may be generally described as having a top, a bottom, and four sides extending between the top and bottom to create a cube-like structure. However, the terminal package 118 may have any suitable shape which encloses the load 120. In some embodiments, the terminal package 118 may be formed from a rigid material such that the terminal package 118 has a rigid or structured shape. Alternatively, the terminal package 118 may be formed from a flexible material such that the terminal package 118 has a flexible shape. Suitable materials for the terminal package 118 include but are not limited to a polymeric non-woven sheet, such as spun-bonded polyethylene (e.g., Tyvek®, sold by E.I. du Pont de Nemours and Company, Wilmington, Del.), and polymeric materials such as polyester and polypropylene. Suitable materials for terminal package 118 having a rigid or structured shape include but are not limited to various metals such as aluminum, stainless steel and/or various polymers in rigid form such as polyethylene and/or polypropylene.

The load 120 may be positioned within the terminal package 118 and subjected to one or more decontamination cycles. Suitable loads for decontamination include lumen devices(e.g., medical, dental or other device) having at least one lumen extending through at least a portion of the device and/or other device or instrument without any lumens. In some embodiments, the load may include at least one lumen extending the entire length of the device. For example, the load 120 may be an endoscope. However, as discussed herein, the load 120 could be any device with zero, one, two or more lumens.

The terminal package 118 may be configured to prevent or reduce microbes and/or other contaminants from entering the terminal package 118. In some embodiments, for example, the terminal package 118 can include a material suitable for allowing flow of a sterilant fluid, such as hydrogen peroxide (H₂O₂) and/or peracetic acid (PAA), into the load receiving area 130 of the terminal package 118 and blocking or reducing the flow of contaminants into the interior of the terminal package 118. In the illustrated embodiment, the terminal package 118 includes a plurality of openings or pores 122 for allowing flow of the sterilant fluid into the terminal package 118. In some embodiments, the pores 122 may be sized so as to allow the sterilant fluid and/or air to communicate into and out of the container 118 as well as prevent microbes from entering the terminal package 118.

In some embodiments, the sterilant fluid can flow from the cassette 150 to decontamination chamber 104 and load 120. In some embodiments, the amount of sterilant fluid introduced into the decontamination chamber 104, the load 120 or a combination thereof can be controlled the size of cavity holding sterilant fluid in the cassette 150. Depending on the circumstances, the cassette 150 could include an electronic valve controlled by the system controller 106 to control the amount and/or rate of the sterilant fluid delivered. In other circumstances, the entire volume of sterilant fluid within a sterilant holding cavity of the cassette could be delivered to the decontamination chamber 104.

To decontaminate a load, such as a medical, dental or other device, the load 120 may be sealed within the terminal package 118 and placed in the decontamination chamber 104; depending on the circumstances, the load 120 could be placed in the decontamination chamber 104 without a terminal package. The load 120 is then subjected to a decontamination process which may include one or more decontamination cycles. The exact decontamination cycle could be based on the profile of the load 120 to be decontaminated. A suitable cycle may include adjusting the pressure of the decontamination chamber 104 to a suitable range, such as to a pressure less than 10 Torr, conditioning using plasma, and introducing the sterilant fluid 132 into the decontamination chamber 104 via the cassette 150. The sterilant fluid may be held within the decontamination chamber 104 for a period of time to facilitate the decontamination of the load 120. After the sterilant fluid has been held in the decontamination chamber 104 for the desired or programmed amount of time, the system controller 106 can vent the decontamination chamber 104 to an atmospheric pressure or a different but sub-atmospheric pressure. The system controller 106 can then hold the pressure within the decontamination chamber 104 for a period of time to further facilitate the decontamination of the load. Following the hold period, the system controller 106 may evacuate the decontamination chamber 104 to remove the sterilant fluid residuals from the decontamination chamber 104 which may also include a plasma treatment to further enhance the removal of the substance residuals, followed by venting the decontamination chamber 104. This cycle or steps may be repeated or extended as part of a comprehensive cycle.

During the decontamination cycle(s), the PCD in the cassette 150 is in fluid communication with the decontamination chamber 104 through a tortuous path defined within the cassette 150. The PCD includes one or more cavities to hold a chemical indicator and/or a biological indicator that is exposed to the decontamination chamber 104 via the tortuous path. The PCD can be read by the PCD monitoring system 112 (or external PCD reader) to determine growth confirmation, which is used to evaluate sterilization effectiveness. The particular position of the PCD monitoring system 112 with regard to the decontamination chamber could vary depending on the circumstances. Typically, however, the PCD monitoring system 112 would be proximate to the cassette 150 to interface, mechanically, electrically, optically, etc., with the indicator 158 and obtain a reading.

FIG. 2 is an example cassette 150 dimensioned to be placed in the cassette receiving area 110 during the decontamination process. In the example shown, the cassette 150 has a cassette body 152 that defines an internal sterilant reservoir 154 with a volume of sterilant fluid 156 and an internal PCD reservoir 157 with an indicator 158 (e.g., chemical and/or biological indicator) that is relevant to determining sterilization effectiveness of the device that has been decontaminated 120. In some embodiments, the chemical and/or biological indicators used comply with one or more of International Organization for Standardization (ISO) standard ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005) or FDA Guidance on Liquid Chemical Sterilants/High Level Disinfectants.

The cassette body 152 could be formed from any materials that form a rigid or structured shape and withstand exposure to the decontamination chamber 104, including but not limited to various metals such as aluminum, stainless steel and/or various polymers in rigid form such as polyethylene and/or polypropylene; likewise, inert, oxidation resistant polymer coatings, such as HDPE, PTFE, PVC, and/or MABS, could be used for the cassette body 152. Typically, the cassette 150 is a consumable and thrown away after the sterilant fluid and indicator have been used; however, in some embodiments, the cassette 150 could be reconditioned with fresh sterilant fluid and indicator.

In some embodiments, the sterilant fluid 156 and/or indicator 158 could be chosen to target certain types of devices to be decontaminated. For example, a certain sterilant and/or indicator may be chosen to target a device based on lumen size of the device to be decontaminated or other factors. For example, a lumen device construction could dictate various parameters of the cassette, such as sterilant fluid and/or indicator and/or tortious path resistance. A device without any lumens could be another load type with different parameters. By way of example, an endoscope with long, narrow lumen(s) could have a targeted biological indicator or a chemical indicator with a tortious path designed with high resistance; in contrast, a mixed load could have a different biological indicator or a chemical indicator and less resistive tortious path that is less challenging. In some embodiments, the parameters could vary based on whether the load is placed in a terminal package 118. For example, depending on the circumstances, the sterilant 156 could be the same, but the tortuous path 164 and/or inlet port 162 could be adjusted to reflect what the terminal package/sterile barrier would add to the load. In some embodiments, the instrument type targeted by the cassette 150 could be visually displayed on the cassette 150 (and/or the cassette's packaging); in this manner, errors in selecting the type of sterilant 156 and/or indicator 158 for a particular device to be decontaminated chosen by technicians operating the system 100 could be reduced. In some cases, there could be a plurality of cassette types that have sterilant and/or indicator selections to target certain lumen devices or other instruments, whether with or without any lumen(s). The technician operating the system 100 would look at the marking on the cassettes 150 (and/or cassettes' packaging) to select the appropriate combination of sterilant and/or indicator for a device to be decontaminated.

The cassette body 152 defines an outlet port 160 in fluid communication with the sterilant fluid 156 and an inlet port 162 in fluid communication with the internal PCD reservoir 157. When the cassette 150 is placed within the cassette receiving area 110, the cassette receiving area 110 is configured such that the outlet port 160 and the inlet port 162 are in fluid communication with the decontamination chamber 104. During the decontamination process, the sterilant fluid 156 would flow out of the outlet port 160 into the decontamination chamber 104 and sterilant fluid 156 from the decontamination chamber 104 would flow into the inlet port 162 through a tortuous path 164 towards the indicator 158.

A valve or other device (not shown) could be placed at the outlet port 160 and inlet port 162 to control flow into/out of the ports 160, 162; for example, the valve could prevent flow of sterilant fluid 156 out of the outlet port 160 except during the decontamination process and prevent exposure of the indicator 158 except during the decontamination process. By way of example only, a check valve could be used that is configured to open at certain pressures corresponding to pressures within the decontamination chamber 104 during the decontamination process; in some embodiments, an electronic valve could be controlled by the system controller 106 to open/close during the decontamination process; in a further embodiment; a frangible material could cover the ports 160, 162 that is configured to burst when exposed to certain pressures during the decontamination process; regardless of the mechanism, the cassette 150 could include any valve-like mechanism that opens during the decontamination process to allow flow of sterilant fluid 156 out of the outlet port 160 into the decontamination chamber 104 and flow into the inlet port 162.

The cassette body 152 defines a tortuous path 164 between the internal PCD reservoir 157 and inlet port 162. The tortuous path 164 limits or restricts flow of sterilant fluid from the decontamination chamber 104 towards the indicator 158 to challenge effectiveness of the decontamination process. The tortuous path 164 could be any path that is indirect or bending (i.e., not straight) between the inlet port 162 and the internal PCD reservoir 157. In some cases, the degree to which the path 164 is tortuous could depend on the type of challenge and/or the type of device to be decontaminated. For example, the shape and/or dimensions of the tortuous path 164 could be designed for targeted loads. The length and/or width of tortuous path 164 would be reflective of the challenge to kill in the worse case location in the load (i.e., the middle of the scope down the channel for an endoscope). By way of another example, a multiple channel scope that has a longer and/or thinner channel would be targeted with a more resistant PCD to mimic that challenge, which could mean the tortuous path 164 could be longer and/or narrower to represent the resistance of the load. For example, the degree to which the path 164 is tortuous could depend on the lumen size and/or length of the device to the decontaminated. In some embodiments, instead (or addition to adjusting the length and/or width of the tortuous path 164), another type of restriction, such as Tyvek® or a sterile barrier system/packaging could be used. In some cases, the cassette 150 could include a rating or indicator on the cassette body (or the cassette's packaging) indicating the degree of challenge posed by the tortuous path 164 and/or device type corresponding to the tortuous path 164.

The embodiment of the cassette 150 shown in FIG. 2 includes a single internal sterilant reservoir 154 and a single internal PCD reservoir 157. This example configuration could be used for a single decontamination load or cycle. In other embodiments, the cassette could be configured for use in more than one load or cycle, and therefore include multiple internal sterilant reservoirs and/or multiple internal PCD reservoirs.

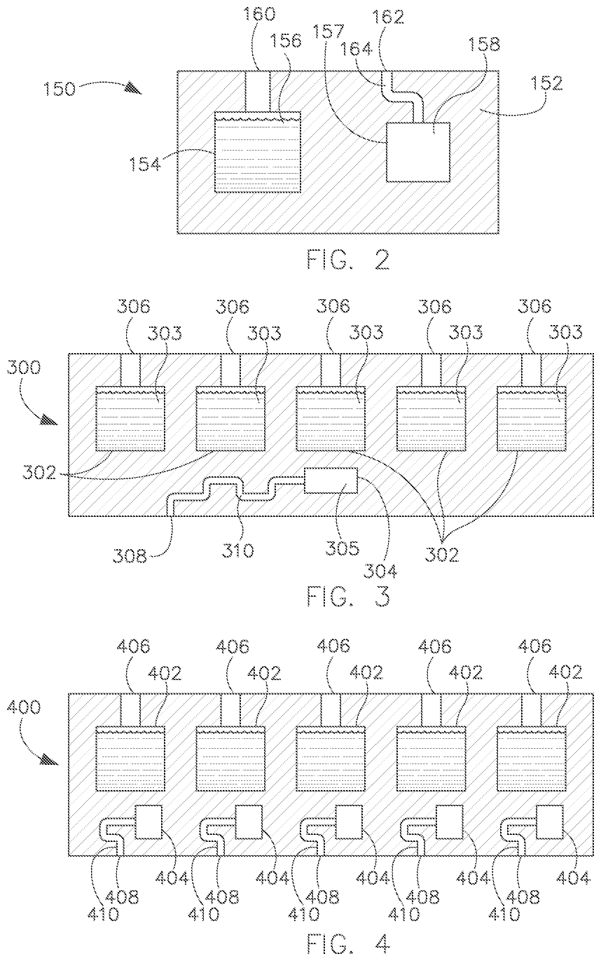

FIG. 3 shows an example cassette 300 with multiple internal sterilant reservoirs 302 with sterilant 303 and a single internal PCD reservoir 304 with a single indicator 305. In this embodiment, there are outlet ports 306 corresponding with each internal sterilant reservoir 302. The cassette 300 includes an inlet port 308 in fluid communication with the internal PCD reservoir 304 via a tortuous path 310. Although five internal sterilant reservoirs 302 are shown for multiple of example, there could be two or more internal sterilant reservoirs 302 in this embodiment depending on how many loads the cassette 300 is intended to last. For example, the cassette 300 could be configured with sufficient internal sterilant reservoirs 302 for an entire day or other period. In this example, the internal PCD reservoir 304 and indicator 305 could be exposed to multiple decontamination loads/cycles. The cassette 300 would function similarly to cassette 150, but the user would select a different internal sterilant reservoir 302 for each decontamination cycle/load. For example, the user could place the cassette 300 in the cassette receiving area 310 such that a selected outlet port 306 is in fluid communication with the decontamination chamber 104.

FIG. 4 shows an example cassette 400 with multiple internal sterilant reservoirs 402 with sterilant 403 and multiple internal PCD reservoirs 404 each with an indicator 405. Although five internal sterilant reservoirs 402 and five internal PCD reservoirs 404 are shown for purposes of example, there could be two or more internal sterilant reservoirs 402 and two or more internal PCD reservoirs 404. In this embodiment, there are outlet ports 406 corresponding with each internal sterilant reservoir 402 and multiple inlet ports 408 corresponding with each internal PCD reservoir 404. A tortuous path 410 extends between each respective inlet port 408 and corresponding internal PCD reservoir 404. In this embodiment, the user would typically select an outlet port and inlet port pair for each decontamination load/cycle. Otherwise, the cassette 400 would function similarly to cassette 150.

### EXAMPLES

Illustrative examples of the method and system disclosed herein are provided below. An embodiment of the method and system may include any one or more, and any combination of, the examples described below.

Example 1 is a decontamination system with a decontamination chamber, a cassette, and a sterilization control system. The decontamination chamber has a plurality of walls defining a device receiving area dimensioned to receive a device to be decontaminated; at least one wall of the plurality of walls define a cassette receiving area in fluid communication with the device receiving area. The cassette is dimensioned to be detachably received by the cassette receiving area, wherein the cassette includes (i) a sterilant delivery portion to deliver a sterilant to the device receiving area; and (ii) a process challenge device (PCD) portion to confirm effective sterilization of the device to be decontaminated, wherein at least a portion of the sterilant delivery portion and at least a portion of the PCD portion is in fluid communication with the device receiving area when the cassette is attached to the cassette receiving area. The sterilization control system is to control sterilant delivery from the sterilant delivery portion to the device receiving area.

In Example 2, the subject matter of Example 1 is further configured such that the cassette includes one or more sterilant reservoirs with one or more of a sterilant or chemical agent within the one or more sterilant reservoirs.

In Example 3, the subject matter of Example 2 is further configured such that the one or more sterilant reservoirs are configured to be in fluid communication with the device receiving area when the cassette is attached to the cassette receiving area.

In Example 4, the subject matter of Example 3 is further configured such that the cassette includes one or more PCD reservoirs each with at least one indicator relevant to determining sterilization effectiveness of the device to be decontaminated.

In Example 5, the subject matter of Example 4 is further configured such that the indicator comprises one or more of a biological indicator or a chemical indicator.

In Example 6, the subject matter of Example 5 is further configured such that the one or more PCD reservoirs are configured to be in fluid communication with the device receiving area.

In Example 7, the subject matter of Example 6 is further configured such that the cassette defines a tortuous path between the indicator and the device receiving area.

In Example 8, the subject matter of Example 1 is further configured to include a plurality of the cassettes, wherein the tortuous path in respective cassettes is configured to be more or less tortuous as a function of the type of device to be decontaminated.

In Example 9, the subject matter of Examples 4-8 are further configured such that the cassette includes a plurality of sterilant reservoirs each with a sterilant or chemical agent within a respective sterilant reservoir of the plurality of sterilant reservoirs.

In Example 10, the subject matter of Example 9 is further configured such that the cassette includes a plurality of PCD reservoirs each with an indicator relevant to sterilization effectiveness of the device to be decontaminated within a respective PCD reservoir of the plurality of PCD reservoirs.

In Example 11, the subject matter of Example 10 is further configured such that the cassette defines separate tortuous paths between each PCD reservoir of the plurality of PCD reservoirs and the device receiving area.

In Example 12, the subject matter of Example 4 is further configured to include a monitoring system configured to read the at least one indicator of the PCD.

In Example 13, the subject matter of Examples 1-12 are further configured such that the cassette is configured for a single sterilization load or sterilization cycle of the device to be decontaminated.

In Example 14, the subject matter of Examples 1-13 are further configured such that the cassette receiving area is defined within a side wall of the decontamination chamber.

Example 15 is a cassette for use with a decontamination system. The cassette includes a cassette body defining at least one internal sterilant reservoir and at least one internal PCD reservoir; a volume of sterilant within the at least one internal sterilant reservoir; at least one indicator relevant to sterilization of a device to be decontaminated within the at least one internal PCD reservoir. The cassette body defines an outlet port to deliver at least a portion of the sterilant from the internal sterilant reservoir and an inlet port to provide fluid communication with the internal PCD reservoir.

In Example 16, the subject matter of Example 15 is further configured such that the cassette body defines a tortuous path between the inlet port and the at least one internal PCD reservoir.

In Example 17, the subject matter of Example 16 is further configured such that the at least one indicator comprises one or more of a biological indicator or a chemical indicator.

In Example 18, the subject matter of Example 17 is further configured to include a plurality of outlet ports each in separate fluid communication with a respective sterilant reservoir of a plurality of sterilant reservoirs, wherein each of the plurality of sterilant reservoirs includes a sterilant or chemical agent.

In Example 19, the subject matter of Example 18 is further configured to include a plurality of inlet ports in separate fluid communication with a respective PCD reservoir of a plurality of PCD reservoirs, wherein each of the plurality of PCD reservoirs includes an indicator relevant to determining effective sterilization of the device to be decontaminated.

In Example 20, the subject matter of Example 19 is further configured such that the cassette body defines a plurality of tortuous fluid paths to establish fluid communication between respective inlet ports and corresponding PCD reservoirs.

Although the present disclosure has been described with reference to particular means, materials and embodiments, from the foregoing description, one skilled in the art can easily ascertain the essential characteristics of the invention and various changes and modifications may be made to adapt the various uses and characteristics without departing from the spirit and scope of the invention.

What is claimed is:

1. A decontamination system comprising:
a decontamination chamber with a plurality of walls defining a device receiving area dimensioned to receive a device to be decontaminated, wherein at least one wall of the plurality of walls define a cassette receiving area in fluid communication with the device receiving area;
a cassette dimensioned to be detachably received by the cassette receiving area, wherein the cassette includes (i) a sterilant delivery portion to deliver a sterilant to the device receiving area; and (ii) a process challenge device (PCD) portion to confirm effective sterilization of the device to be decontaminated, wherein at least a portion of the sterilant delivery portion and at least a portion of the PCD portion is in fluid communication with the device receiving area when the cassette is attached to the cassette receiving area; and
a sterilization control system to control sterilant delivery from the sterilant delivery portion to the device receiving area.

2. The decontamination system of claim 1, wherein the cassette includes one or more sterilant reservoirs with one or more of a sterilant or chemical agent within the one or more sterilant reservoirs.

3. The decontamination system of claim 2, wherein the one or more sterilant reservoirs are configured to be in fluid communication with the device receiving area when the cassette is attached to the cassette receiving area.

4. The decontamination system of claim 3, wherein the cassette includes one or more PCD reservoirs each with at least one indicator relevant to determining sterilization effectiveness of the device to be decontaminated.

5. The decontamination system of claim 4, wherein the indicator comprises one or more of a biological indicator or a chemical indicator.

6. The decontamination system of claim 5, wherein the one or more PCD reservoirs are configured to be in fluid communication with the device receiving area.

7. The decontamination system of claim 6, wherein the cassette defines a tortuous path between the indicator and the device receiving area.

8. The decontamination system of claim 7, further comprising a plurality of the cassettes, wherein the tortuous path in respective cassettes is configured to be more or less tortuous as a function of the type of device to be decontaminated.

9. The decontamination system of claim 4, wherein the cassette includes a plurality of sterilant reservoirs each with a sterilant or chemical agent within a respective sterilant reservoir of the plurality of sterilant reservoirs.

10. The decontamination system of claim 9, wherein the cassette includes a plurality of PCD reservoirs each with an indicator relevant to sterilization effectiveness of the device to be decontaminated within a respective PCD reservoir of the plurality of PCD reservoirs.

11. The decontamination system of claim 10, wherein the cassette defines separate tortuous paths between each PCD reservoir of the plurality of PCD reservoirs and the device receiving area.

12. The decontamination system of claim 4, further comprising a monitoring system configured to read the at least one indicator of the PCD.

13. The decontamination system of claim 1, wherein the cassette is configured for a single sterilization load or sterilization cycle of the device to be decontaminated.

14. The decontamination system of claim 1, wherein the cassette receiving area is defined within a side wall of the decontamination chamber.

15. A cassette for use with a decontamination system, the cassette comprising:
a cassette body defining at least one internal sterilant reservoir and at least one internal PCD reservoir;
a volume of sterilant within the at least one internal sterilant reservoir;
at least one indicator relevant to sterilization of a device to be decontaminated within the at least one internal PCD reservoir;
wherein the cassette body defines an outlet port to deliver at least a portion of the sterilant from the internal sterilant reservoir and an inlet port to provide fluid communication with the internal PCD reservoir.

16. The cassette of claim 15, wherein the cassette body defines a tortuous path between the inlet port and the at least one internal PCD reservoir.

17. The cassette of claim 16, wherein the at least one indicator comprises one or more of a biological indicator or a chemical indicator.

18. The cassette of claim 17, further comprising a plurality of outlet ports each in separate fluid communication with a respective sterilant reservoir of a plurality of sterilant reservoirs, wherein each of the plurality of sterilant reservoirs includes a sterilant or chemical agent.

19. The cassette of claim 18, further comprising a plurality of inlet ports in separate fluid communication with a respective PCD reservoir of a plurality of PCD reservoirs, wherein each of the plurality of PCD reservoirs includes an indicator relevant to determining effective sterilization of the device to be decontaminated.

20. The cassette of claim 19, wherein the cassette body defines a plurality of tortuous fluid paths to establish fluid communication between respective inlet ports and corresponding PCD reservoirs.
